(19) United States
(12) Patent Application Publication
(10) Pub. No.: US 2008/0058922 A1
(43) Pub. Date: Mar. 6, 2008

(54) METHODS AND DEVICES EMPLOYING VAP-1 INHIBITORS

(75) Inventor: Craig Stolen, New Brighton, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/469,064

(22) Filed: Aug. 31, 2006

(51) Int. Cl. A61F 2/06

(52) U.S. Cl. 623/1.42

(57) ABSTRACT

An implantable device which employs inhibitors of VAP-1, e.g., inhibitors that reversibly or irreversibly bind to VAP-1, and methods of using the devices is provided.

### FIELD OF THE INVENTION

This invention relates generally to therapy of living tissue which employs, but not by way of limitation, an implantable device and agents that inhibit one or more adhesion molecules, e.g., VAP-1.

### BACKGROUND

The normal human heart is a strong, muscular pump a little larger than a fist. It pumps blood continuously through the circulatory system. Each day the average heart “beats” (or expands and contracts) 100,000 times and pumps about 2,000 gallons of blood. In a 70-year lifetime, an average human heart beats more than 2.5 billion times.

The heart pumps blood through a circulatory system, which is a network of elastic tubes through which blood flows as it carries oxygen and nutrients to all parts of the body. The circulatory system includes the heart, lungs, arteries, arterioles (small arteries), and capillaries (minute blood vessels). It also includes venules (small veins) and veins, the blood vessels through which blood flows as it returns to the heart.

The circulating blood brings oxygen and nutrients to all the organs and tissues of the body, including the heart itself. The blood also picks up waste products from the body's cells. These waste products are removed as they're filtered through the kidneys, liver and lungs.

Over time, the coronary arteries which supply the heart muscle with blood can become clogged. One cause of clogged arteries is due to a condition called atherosclerosis, or hardening of the arteries. Atherosclerosis causes a constriction of the inner lumen of the affected artery when the lumen of the arteries become more narrow due to a pathological accumulation of cells, fats and cholesterol called plaque. The descriptive term given to this narrowing of the coronary arteries is “stenosis.” Stenosis means constriction or narrowing. A coronary artery that is constricted or narrowed is referred to as stenosed. When stenosis of the coronary artery is sufficient to deprive the heart muscle of the oxygen levels necessary for cell viability, the result is typically myocardial infarction, typically referred to as a heart attack.

A heart attack occurs when the blood supply to part of the heart muscle itself, the myocardium, ceases or is severely reduced. This occurs when one or more of the arteries supplying blood to the heart muscle (coronary arteries) become partially or completely obstructed by plaque stenoses. If cessation of the blood supply occurs for a long time, heart muscle cells suffer irreversible injury and die. Severe disability or death can result, depending on how much heart muscle is damaged.

Coronary artery bypass surgery is a heart operation used to treat coronary artery disease. In coronary artery bypass surgery a blood vessel is used to go around or “bypass” clogged coronary (heart) arteries. During the bypass procedure, a blood vessel from the patient's chest or leg is used as the “bypass” conduit. For venous “bypass” grafts, one end of the vessel is attached to the aorta (the large artery coming out of the heart) and the other end is attached to the coronary artery below the point where it's clogged. Once the clog has been bypassed, blood can once again flow through the bypass graft to the heart, in a manner that prevents ischemia and infarction. Almost half a million coronary bypass operations are performed each year in the USA.

Another procedure for opening clogged coronary arteries is to perform percutaneous transluminal coronary angioplasty, or balloon angioplasty. Balloon angioplasty is an established and effective therapy for some patients with coronary artery disease. Balloon angioplasty is used to dilate (widen) arteries narrowed by plaque. During the procedure, a catheter with a deflated balloon on its tip is passed into the narrowed part of the artery. The balloon in then inflated, and the narrowed area is widened. Balloon angioplasty is a less traumatic and less expensive alternative to bypass surgery for some patients with coronary artery disease. However, in 25 to 30 percent of patients the dilated segment of the artery renarrows (restenosis) within six months after the procedure. The patient may then require either to repeat the balloon angioplasty or to undergo coronary bypass surgery.

One approach to preventing restenosis has been to insert a stent across the stenosed area of coronary artery. A stent is a metallic wire mesh tube that is used to prop open an artery that has been recently dilated using balloon angioplasty. The stent is collapsed to a small diameter, placed over an angioplasty balloon catheter and moved into the area of the blockage. When the balloon is inflated, the stent expands, locking in place to form a rigid support (structural scaffolding) which holds the artery lumen open. The stent remains in the artery permanently to help improve blood flow to the heart muscle. However, reclosure (restenosis) remains an important issue with the stent procedure.

Several approaches have been taken to reduce the occurrence of restenosis associated with the stent procedure. Stents have been impregnated with drugs and chemicals that emit radiation (gamma-rays) in an attempt to reduce the frequency of restenosis. Also, drug eluting stents have been used in an attempt to reduce the occurrence of restenosis. However, a need still exists for additional safe and effective treatments to prevent restenosis after the placement of an intravascular stent.

What is needed is an improved method to inhibit inflammation and restenosis.

### SUMMARY OF THE INVENTION

The invention provides methods which employ agents that inhibit or block binding of endogenous circulating cells, e.g., stem cells, to adhesion molecules, thereby decreasing endogenous cell implantation at a selected site. For example, vascular adhesion protein-1 (VAP-1) inhibitors, e.g., semicarbazide sensitive amine oxidase (SSAO) inhibitors, may be employed. The delivery of VAP-1 inhibitors, e.g., via a stent, in an effective amount may inhibit inflammation, restenosis, oxidative stress, e.g., reactive oxygen species (ROS) production, or a combination thereof. In one embodiment, the VAP-1 inhibitor may be an antibody, e.g., a humanized, chimeric, or ScFv antibody. In another embodiment, the VAP-1 inhibitor is not an antibody, for instance, the VAP-1 inhibitor is a drug containing a hydrazine, arylalkylamine, propenylamine, proparylamine, oxazolidinone or haloalkylamine. In one embodiment, one or more VAP-1 inhibitors are combined with a carrier such as a polymer, phosphoryleholine, or a ceramic, to provide for sustained release of the one or more inhibitors. In one embodiment, the one or more VAP-1 inhibitors are administered along with an immunosuppressive and/or an antiproliferative, for instance, sirolimus (rapamycin), paclitaxel, zotarolimus or everolimus. The immunosuppressive or antiproliferative may be administered separately or via the same delivery vehicle as the VAP-1 inhibitor, e.g., via the same stent or lead, to the same mammal.

Thus, the invention provides an implantable device for treating living tissue. The device includes an intravascular device and a composition comprising one or more agents incorporated into at least a portion of the intravascular device, wherein the one or more agents include a VAP-1 inhibitor.

Further provided is a method to inhibit or treat inflammation. The method includes introducing to a mammal in need thereof an implantable device comprising a composition comprising an effective amount of one or more agents incorporated into at least a portion of the intravascular device, wherein the one or more agents include a VAP-1 inhibitor.

In one embodiment, the invention provides a method to inhibit or treat restenosis. The method includes introducing to a mammal in need thereof an implantable device comprising a composition comprising an effective amount of one or more agents incorporated into at least a portion of the intravascular device, wherein the one or more agents include a VAP-1 inhibitor. In one embodiment, diabetics are at increased risk for restenosis after coronary angioplasty stenting. For example, diabetics having increased VAP-1 levels may particularly benefit from placement of a stent having one or more VAP-1 inhibitors.

In another embodiment, a method to inhibit or treat oxidative stress in a mammal is provided. The method includes introducing to a mammal in need thereof an implantable device comprising a composition comprising an effective amount of one or more agents incorporated into at least a portion of the intravascular device, wherein the one or more agents comprise a VAP-1 inhibitor.

### BRIEF DESCRIPTION OF THE FIGURES

FIG. 1 is an illustration of an embodiment of an agent on a surface portion of a device.

FIG. 2 is an illustration of an embodiment of an intravascular device including an agent.

FIG. 3 is an illustration of an embodiment of an implantable lead.

FIG. 4 is an illustration of an embodiment of an implantable lead with a distal end including a coating carrying an agent.

FIG. 5 is an illustration of an embodiment of a stent carrying an agent mounted on an expandable member of a conventional catheter assembly.

FIG. 6 is an illustration of an embodiment of a stent and expandable member in an expanded state.

FIG. 7 is an illustration of an embodiment of a stent with an expandable member removed.

### DETAILED DESCRIPTION OF THE INVENTION

### DEFINITIONS

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. An “animal” includes vertebrates such as mammals, avians, amphibians, reptiles and aquatic organisms including fish.

As used herein, “adhesion molecules” include but are not limited to selectins, e.g., L-selectin, E-selectin and P-selectin, mucines, integrins, e.g., LFA-1 and ICAM-1, Ig superfamily members, VAP-1, retina-specific amine oxidase (AOC2), ectoenzymes, and ligands thereof. For instance, VLA-4 (an integrin) binds VCAM (CD106), LFA-1 (an integrin, CD11/18) binds ICAM (CD54), L-selectin (CD62) binds CD34, and CD44 binds hyaluronan (HA).

The terms “effective amount” or “amount effective to” or “therapeutically effective amount” refers to an amount sufficient to induce a detectable therapeutic response in the subject. Assays for determining therapeutic responses are well known in the art. For example repair (i.e., healing) of injured myocardium can be detected using magnetic resonance imaging (MRI) to detect changes in the myocardium that are indicative of tissue regrowth and reformation.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

The terms, “patient”, “subject” or “animal” are used interchangeably and refer to a mammalian subject to be treated, with human patients being preferred In some cases, the methods of the invention find use in experimental animals, in veterinary application, and in the development of animal models for disease, including, but not limited to, rodents including mice, rats, and hamsters; and primates.

As used herein, “pharmaceutically acceptable salts” refer to derivatives of compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou et al. (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

A compound can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein carbonyl, carboxylic acid, hydroxy or amino groups are bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

### GENERAL OVERVIEW

This document describes, among other things compositions, methods and devices to inhibit or treat restenosis, inflammation, oxidative stress, or a combination thereof, and/or inhibit endogenous cell implantation at a selected physiological site. In one embodiment, an agent that inhibits expression, activation or activity of the adhesion molecule VAP-1 may be employed. The agent may be administered systemically or locally (e.g., via injection, stent or catheter delivery).

The invention thus provides methods to inhibit homing and/or extravasation of endogenous cells by administering an agent that blocks or inhibits binding of the adhesion molecules to a target tissue. For example, small molecule inhibitors or antibodies to adhesion molecules such as antibodies to VAP-1, selecting, or integrins, may be applied to a tissue, e.g., locally, to prevent or inhibit unwanted endogenous stem cell homing. In one embodiment, an agent that blocks or inhibits adhesion molecules may be incorporated into a stent to prevent or reduce restenosis, e.g., to block or inhibit smooth muscle cells derived from circulating stem cells. For instance, a sustained release form of one or more VAP-1 inhibitors is applied to or incorporated in a stent, e.g., a metal or biodegradable stent, in an amount effective to prevent or reduce restenosis. In another embodiment, an adhesion inhibiting agent may be injected into a tumor (e.g., to prevent or inhibit angiogenesis). In yet another embodiment, an adhesion inhibiting agent may be injected into a heart (e.g., to prevent or inhibit cardiomyopathy or scarring). Thus, the invention may be useful to inhibit or treat many conditions including but not limited to myocardial infarction, heart failure, cardiomyopathy, restenosis, cancer and other diseases.

### BIOCOMPATIBLE MATERIALS FOR USE WITH AGENTS OF THE INVENTION

The agents of the invention may be coated on and/or embedded in a biocompatible material which in turn may be coated on and/or embedded in a device. Biocompatible materials include polyacetic or polyglycolic acid and derivatives combinations thereof.

Additionally, it is possible to construct biocompatible materials from natural materials such as proteins or materials which may be crosslinked using a crosslinking agent such as 1-ethyl-3-(3-dimethylamino-propyl)carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or polymers having pendant zwitterionic groups, specifically phosphorylcholine (PC) groups, generally described in WO 93/01221, or those described in WO 98/30615. Polymers may have pendant crosslinkable groups which are subsequently crosslinked by exposure to suitable conditions, generally heat and/or moisture.

In one embodiment, the material may include liposomes, a hydrogel, cyclodextrins, nanocapsules or microspheres. Thus, a biocompatible material includes synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., Mol. Ther., 7:401 (2003)), poly orthoesters (Heller et al., Adv. Drug Delivery Rev., 54:1015 (2002)), silk-elastin-like polymers (Megeld et al., Pharma. Res., 19:954 (2002)), alginate (Wee et al., Adv. Drug Deliv. Rev., 31:267 (1998)), EVAc (poly(ethylene-co-vinyl acetate), microspheres such as poly (D,L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, the biocompatible material includes but is not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols.

In some embodiments, the biocompatible polymeric material is a biodegradable polymeric such as collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), poly(dioxanone) (PPS) or cellulose derivatives such as cellulose acetate. In an alternative embodiment, a biologically derived polymer, such as protein, collagen, e.g., hydroxylated collagen, or fibrin, or polylactic-polyglycolic acid or a polyanhydride, is a suitable polymeric matrix material.

In another embodiment, the biocompatible material includes polyethyleneterephalate, polytetrafluoroethylene, copolymer of polyethylene oxide and polypropylene oxide, a combination of polyglycolic acid and polyhydroxyalkanoate, or gelatin, alginate, collagen, hydrogels, poly-3-hydroxybutyrate, poly-4-hydroxybutyrate, and polyhydroxyoctanoate, and polyacrylonitrilepolyvinylchlorides.

Other biocompatible materials include natural polymers such as starch, chitin, glycosaminoglycans, e.g., hyaluronic acid, dermatan sulfate and chrondrotin sulfate, collagen, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone. The incorporation of molecules such as tricalciumphosphate, hydroxyapetite and basic salts into a polymer matrix can alter the degradation and resorption kinetics of the matrix. Moreover, the properties of polymers can be modified using cross-linking agents.

In one embodiment, the biocompatible material is isolated ECM. ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like. The preparation and use of isolated ECM in vivo is described in co-pending, commonly assigned U.S. patent application Ser. No. 11/017,237, entitled “USE OF EXTRACELLULAR MATRIX AND ELECTRICAL THERAPY,” filed on Dec. 20, 2004, which is hereby incorporated by reference in its entirety.

In one embodiment, the agent may be encapsulated in a sustained delivery vehicle such as, but not limited to, a liposome or an absorbable polymeric particle. The preparation and use of such sustained delivery vehicles are well known to those of ordinary skill in the art. The sustained delivery vehicle containing the agent is then suspended in the composition.

Bioerodible microspheres can be prepared using any of the methods developed for making microspheres for drug delivery, for example, as described by Mathiowitz and Langer, J. Controlled Release, 5:13 (1987); Mathiowitz et al., Reactive Polymers, 6:275 (1987); and Mathiowitz et al., J. Appl. Polymer Sci., 35:755 (1988), the teachings of which are hereby incorporated by reference. The selection of the method depends on the polymer selection, the size, external morphology, and crystallinity that is desired, as described, for example, by Mathiowitz et al., Scanning Microscopy, 4:329 (1990); Mathiowitz et al., J. Appl. Polymer Sci., 45:125 (1992); and Benita et al., J. Pharm. Sci., 73:1721 (1984), the teachings of which are incorporated herein

### EXEMPLARY COMPOSITIONS

The present invention also relates to a pharmaceutical composition including one or more agents that inhibit expression, activation or activity of adhesion molecules in endogenous tissue, in a pharmaceutically acceptable carrier. In one embodiment, the pharmaceutical composition includes agents that inhibit or block expression, activation or activity of adhesion molecules in endogenous tissue. In some therapeutic applications, compositions are administered to a patient suffering from a disease (e.g., cardiovascular disease), in an amount sufficient to cure or at least partially arrest the disease and its complications, e.g., by repairing injured myocardium or reducing occlusion in vessels. An amount adequate to accomplish this is defined as a therapeutically effective dose. Amounts effective for this use depend on the severity of the cardiovascular disease and the general state of the patient's health.

The amount of the agent, or an active salt or derivative thereof, required for use alone or with other compounds will vary not only with the particular salt selected but also with the route of administration, the nature of the condition being treated and the age and condition of the patient and will be ultimately at the discretion of the attendant physician or clinician. In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The pharmaceutical compositions of the present invention may be administered by any means known in the art. Preferably, the compositions are suitable for parenteral administration (e.g., intravenous, intraperitoneal). The compositions of the invention may also be administered subcutaneously, into vascular spaces, or into joints, e.g., intraarticular injection.

Single or multiple administrations of the compositions may be administered depending on the dosage and frequency as required and tolerated by the patient. In any event, the composition should provide a sufficient quantity of the agent to effectively treat the patient, e.g., to repair or augment repair of injured myocardium.

Preferably, the compositions for administration include a solution of the composition and a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, sterilization techniques known in the art. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The composition having an agent that inhibits expression, activation or activity of adhesion molecules may also formulated in microspheres, liposomes or other microparticulate delivery systems. The concentration of composition having an agent that inhibits expression, activation or activity of adhesion molecules in these formulations can vary widely, and will be selected primarily based on fluid volumes, viscosities, body weight and the like in accordance with the particular mode of administration selected and the patient's needs.

The pharmaceutical compositions having an agent that inhibits expression, activation or activity of adhesion molecules may be administered in a therapeutically effective dose over either a single day or several days by daily intravenous infusion. The dose will be dependent upon the properties of the composition having an agent that inhibits expression, activation or activity of adhesion molecules employed, e.g., its activity and biological half-life, the concentration of the composition having an agent that inhibits expression, activation or activity of adhesion molecules in the formulation, the site and rate of dosage, the clinical tolerance of the patient involved, the extent of disease afflicting the patient and the like as is well within the skill of the physician.

The compositions may be administered in solution. The pH of the solution should be in the range of pH 5 to 9.5, preferably pH 6.5 to 7.5. The compositions thereof should be in a solution having a suitable pharmaceutically acceptable buffer such as phosphate, tris (hydroxymethyl) aminomethane-HCl or citrate and the like. Buffer concentrations should be in the range of 1 to 100 mM. The solution of the compositions may also contain a salt, such as sodium chloride or potassium chloride in a concentration of 50 to 150 mM. An effective amount of a stabilizing agent such as albumin, a globulin, a detergent, a gelatin, a protamine or a salt of protamine may also be included and may be added to a solution containing the agent or to the composition from which the solution is prepared. In some embodiments, systemic administration of the composition is typically made every two to three days or once a week. Alternatively, daily administration is useful.

The compositions described herein can be administered to a patient in conjunction with other therapies, e.g., therapies for cardiovascular disease. For example, the compositions may be administered in conjunction with angioplasty to promote repair of injured cardiac tissue. The compositions may be administered prior to the angioplasty, contemporaneous with the angioplasty, or subsequent to the angioplasty. In one embodiment, the composition is delivered via a stent, e.g., from a sustained released formulation coating and/or embedded in the stent. In another embodiment, VAP-1 inhibitors may be administered at the time of stenting, e.g., via oral, IV or catheter administration.

For example, a VAP-1 inhibitor may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

### Agents and Treatments Useful in the Methods and Devices

Agents and treatments useful in the methods of the invention include those which inhibit cell surface molecule expression, activation or activity of adhesion molecules or their ligands (see Table 1) on target tissue or cells, e.g., ex vivo, at a particular physiological site, or both, and including neutralizing and antagonistic antibodies of adhesion molecules or their ligands.

TABLE 1

| Adhesion molecules | Other names | Ligands |
|---|---|---|
| Selectins/ligands | | |
| P-selectin | CD62P, GMP140 | PSGL-1, Lewis X, CD24 |
| E-selectin | CD62D, ELAM1 | ESL-1, Lewis X, PSGL-1, |
| | | Lyset |
| L-selectin | CD62L | Lewis X, CD 34, PSGL-1, |
| | | GlyCAM |
| E-selectin ligand 1 | ESL-1 | E-selectin |
| P-selectin ligand 1 | CD162, PSGL-1 | P-, L-, E-selectin |
| | | PNAd, cutaneous |
| | | lymphocyte antigen |
| | | (CLA), CD15 (Sialyl- |
| | | Lewis X) |
| Ectoenzymes and other | | |
| adhesion molecules | | |
| VAP-1 | semicarbazide-sensitive | amine groups |
| | amino oxidase (SSAO), | |
| | AOC-3, HPAO, and | |
| | membrane, copper amine oxidase | |
| retina-specific amine | AOC2 | amine groups |
| oxidase | | |
| CD26 | EC3.4.14.5, adenosine | adenosine deaminase, |
| | deaminase binding | collagen, CD45 |
| | protein, ADA binding | |
| | protein, | |
| | dipeptidylpeptidase IV, | |
| | DPPIV ectoenzyme | |
| CD38 | T10, ADP- | CD31, hyaluronic acid |
| | ribosylcyclase; cyclic | |
| | ADP-ribose hydrolase | |
| CD73 | Ecto-5′-nucleotidase | |
| mannose receptor | | collagen |
| clever-1 (common | stabilin-1, FEEL-1 | |
| lymphatic endothelial and | | |
| vascular receptor-1) | | |
| CD40 | Bp50 | CD40L |
| CD44 | ECMRIII, HCAM, | Hyaluronan, MIP-1β, |
| | HUTCH-1, Hermes, Lu, | osteopontin, ankyrin, |
| | In-related, Pgp-1, gp85 | fibronectin |
| Immunoglobulins | | |
| ICAM-1 | CD54 | αLβ2, αMβ, αXβ2 |
| ICAM-2 | CD102 | αLβ2, αMβ |
| ICAM-3 | CD50 | αLβ2, αDβ2, DC-SIGN |
| VCAM-1 | CD106 | α4β1, α4β7 αDβ2 |
| PECAM-1 | CD31 | PECAM-1, Vβ3 |
| NCAM-1 | LFA-3 (lymphocyte | CD2 |
| | function associated | |
| | antigen-3), CD58 | |
| MAdCAM-1 (mucosal | MACAM-1, mucosal | |
| vascular addressin cell | addressin cell adhesion | |
| adhesion molecule-1) | molecule-1 precursor | |
| JAM-2 (junctional | C21orf43, HGNC1284, | |
| adhesion molecule-2), | JAMA-A, JAM-B, | |
| | Junctional adhesion | |
| | molecule B precursor, | |
| | PRO245, | |
| | UNQ219/PRO245, | |
| | vascular endothelial | |
| | junction-associated | |
| | molecule, VE-JAM, | |
| | CD322 | |
| JAM-1 (junctional | Jcam-1, JAM-A, Jcam, | |
| adhesion molecule-1) | Junctional adhesion | |
| | molecule A precursor, | |
| | F11 receptor, Ly106, | |
| | AA638916, 913004G24, | |
| | BV11 antigen, | |
| | ESTM33, CD321 | |
| Mucins | | |
| Mad-CAM-1 | | α4β7 integrin, L-selectin |
| GlyCAM-1 (glycosylation | | L-selectin |
| dependent cell adhesion | | |
| molecule-1) | | |
| Integrins | | |
| Integrin α2/β1 | CD49b/CD29, VLA2 | Collagen, laminin |
| Integrin α4/β1 | CD49d/CD29, VLA4 | VCAM-1, FN |
| Integrin αL/β2 | CD11a/CD18, LFA1 | ICAMs |
| Integrin αM/β2 | CD11b/CD18, Mac1 | ICAMs, iC3b FX, FG |
| Integrin αX/β2 | CD11c/CD18 | ICAM-1, FG, iC3b, CD23 |
| Integrin αD/β2 | CD11d/CD18 | ICAM-3, VCAM-1 |
| Integrin α2B/α3 | GPIIb/IIIa | vWF, FN, FG, VN, |
| | | thrombospondin |
| Integrin αV/β3 | VNR, CD51/CD61 | PECAM-1, WN, FN, FG, |
| | | vWF, VN |
| Integrin αV/β5 | | |
| Integrin αA/β7 | | |

Agents useful to inhibit localization of endogenous circulating stem cells, or to inhibit or treat inflammation, restenosis or oxidative stress, include inhibitors of VAP-1 or SSAO, e.g., hydrazine derivatives, e.g., aryl(alkyl)hydrazines, arylalkylamines, propenyl- and proparyl-amines, oxazolidinones and haloalkylamines, including but not limited to 3-halo-2-phenylallylamines, semicarbazide, hydroxylamine, propargylamine, pyridoxamine, (+)mexiletine, B-24 (3,5-diethoxy-4-aminomethylpyridine), amiflamine (FLA 336(+)), FLA336(−), FLA788(+), FLA668(+), MDL-72145 ((E)-2-(3,4-dimethyloxyphenyl)-3-fluoroallyamine, MDL-72974A ((E)-2-(4-fluorophenethyl)-3-fluoroallylamine hydrochloride), iproniazid, phenelzine, procarbazine (N-isopropyl-alpha-(2-methylhydrazino)-p-toluamide hydrochloride), hydralazine, carbidopa, benserazide, aminoguanidine (pimagedine), 2 bromoethylamine, and carbocyclic hydrazino compounds, or a pharmaceutically acceptable salt thereof.

In one embodiment, inhibitors of VAP-1 or other copper containing amine oxidases such as SSAO useful in the devices and methods of the invention include but are not limited to those disclosed in U.S. Pat. Nos. 6,982,286, 6,624,202, and 6,066,321; U.S. published application 20060128770 (thiazole derivatives), 20060025438, 20050096360, 20040259923, 20040236108 (carbocyclic hydrazine), 20040106654, 20030125360, 20020173521, and 20020198189; Koskinen et al. (Blood, 103:3388 (2004)), Lazar et al. (Acta Pharma. Hungarica, 74:11 (2004), peptide inhibitors in Yegutkin et al. (Eur. J. Immunol., 34:2276 (2004)), Wang et al. (J. Med. Chem., 49:2166 (2006)), e.g., compounds 4a and 4c therein, esterified pectins such as those disclosed in Hou et al. (J. Ag. Food Chem., 51:6362 (2003)), e.g., DE65T4, DE94T18, DE25T4, and DE94T4, and includes anti-VAP antibodies such as those described in U.S. Pat. Nos. 5,580,780 and 5,512,442, and Koskinen et al. (Blood, 103:3388 (2004)), Arvilommi et al. (Eur. J. Immunol., 26:825 (1996)), Salmi et al. (J. Exp. Med., 178:2255 (1993)), and Kirten et al. (Eur. J. Immunol., 35:3119 (2005)). Other inhibitors of VAP-1 include, but are not limited to, phenylhydrazine, 5-hydroxytryptamine, 3-bromopropylamine, N-(phenyl-allyl)-hydrazine HCl (LJP-1207), 2-hydrazinopyridine, TNF-α, MDL-72274 ((E)-2-phenyl-3-chloroallylamine hydrochloride), MDL-72214 (2-phenylallylamine), mexiletine, isoniazid, an endogeneous molecule, e.g., see Lizcano et al., J. Neurol. Trans., 32:323 (1990) including one about 500 to 700 MW (see Obata et al., Neurosci. Lett., 296:58 (2000)), imipramine, maprotiline, zimeldine, nomifensine, azoprocarbazine, monomethylhydrazine, d1-alpha methyltryptamine, dl-alpha methylbenzylamine, MD780236 (Dostert et al., J. Pharmacy & Pharmacol., 36:782 (2984)), 2-(dimethyl(2-phenylethyl)silyl) methanamine, cuprozine, alkylamino derivatives of 4-amniomethylpyridine (Bertini et al., J. Med. Chem., 48:664 (2005)), and kynuramine. Preferred inhibitors are selective SSAO inhibitors, e.g., agents that inhibit SSAOs at least 2-fold more than MAOs. Inhibitors may be reversible, competitive, noncompetitive or irreversible inhibitors.

### Exemplary Devices For Agents that Alter Expression of Adhesion Molecules

Devices useful for administering agents to an organ or body part, include a lumen, and may be, but are not limited to, a catheter, needle, stent, e.g., be made of stainless steel, Nitinol (NiTi), or chromium alloy and biodegradable materials, a stent graft, a synthetic vascular graft, e.g., one made of a cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials, a pacemaker, lead, e.g., pacemaker lead, defibrillator, a hemodialysis catheter, or a drug delivery port. The medical device can be made of numerous materials depending on the device. In one embodiment, the device is coated with one or more agents. For example, adhesion molecules or peptides thereof may be coated on the inside lumen of a catheter via a linker, e.g., polyethylene glycol (PEG) based linker. Thus, as cells are delivered to a mammal, they are activated by interaction with the adhesion molecules or peptides thereof that are coated on the lumen of the catheter.

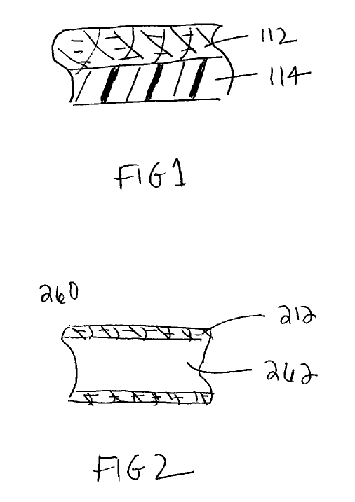

FIG. 1 is an illustration of an embodiment of an agent 112 on a surface portion 114 of a device. In various embodiments, one or more agents 112 are coated on surface portion 114. In another embodiment, one or more agents are released, e.g., via passive or active means, from surface portion 114. In a specific embodiment, the one or more inhibitory agents block the adhesion of circulating endogenous cells. Examples of such inhibitory agents include but are not limited to a VAP-1 inhibitor, such as a SSAO inhibitor. In a specific embodiment, the one or more inhibitory agents include an agent that inhibits or prevents restenosis.

Surface portion 114 is the portion of the device including a surface on which agent 112 is coated or otherwise formed. In one embodiment, the device is a percutaneous injection device that allows for injection of agent 112, such as a delivery catheter, a syringe, or a needle. In another embodiment, the device is an implantable device such as a bead, a transvascular lead, an intravascular stent, an implantable sensor, an implantable pulse generator (such as a pacemaker, a defibrillator, and a neurostimulator), and any other implantable device that delivers electrical, drug, and/or biologic therapies. The implantable device includes a portion that is a surface portion 114.

FIG. 2 is an illustration of an embodiment of a portion of an intraluminal device 260. Intraluminal device 260 includes any device that includes at least a portion configured to be placed in a body lumen to perform sensing and/or therapeutic functions. A surface portion 262 of intraluminal device 260 includes one or more inhibitory agents 212 that inhibit localization and/or extravasation of endogenous circulating cells. One or more inhibitory agents 212 block the adhesion of the endogenous circulating cells. In one embodiment, the one or more inhibitory agents include an agent that inhibits or prevents restenosis, which agent is coated on at least a portion of the coronary stent. Examples of such inhibitory agents include a VAP-1 inhibitor, for instance, a SSAO inhibitor. Specific examples of SSAO inhibitors include but are not limited to semicarbazide, hydroxylamine, propargylamine, pyridoxamine, (+)mexiletine, B-24, FLA 336, MDL-72145, MDL-72974A, iproniazid, phenelzine, procarbazine, hydralazine, carbidopa, benserazide, aminoguanidine, and 2 bromoethylamine, and carbocyclic hydrazine compounds.

In one embodiment, intraluminal device 260 is an intravascular device including at least a portion coated with one or more inhibitory agents 212. Examples of the intravascular device include a stent and a transvenous lead. In a specific embodiment, the intravascular device is a coronary stent. In other specific embodiments, the intravascular device is a device for one or more peripheral vascular applications including, but not limited to, aortic aneurysm repair, carotid artery stenting, iliac artery stenting, femoropopliteal stenting, infrapopliteal stenting, renal artery stenting, and transjugular intrahepatic portosystemic shunting. In another embodiment, the intravascular device is a transvenous pacing and/or defibrillation lead. In another embodiment, intraluminal device 260 includes a non-vascular device, such as a non-vascular stent, including at least a portion coated with one or more inhibitory agents 212. The non-vascular device is configured to be placed in a body lumen that is external to the vascular system. In various specific embodiments, the non-vascular device is configured to be placed in a non-vascular lumen such as biliary, tracheobronchial, oesophagus, upper gastrointestinal tract, the small bowel, large bowel, or dacryocystic duct.

### Leads

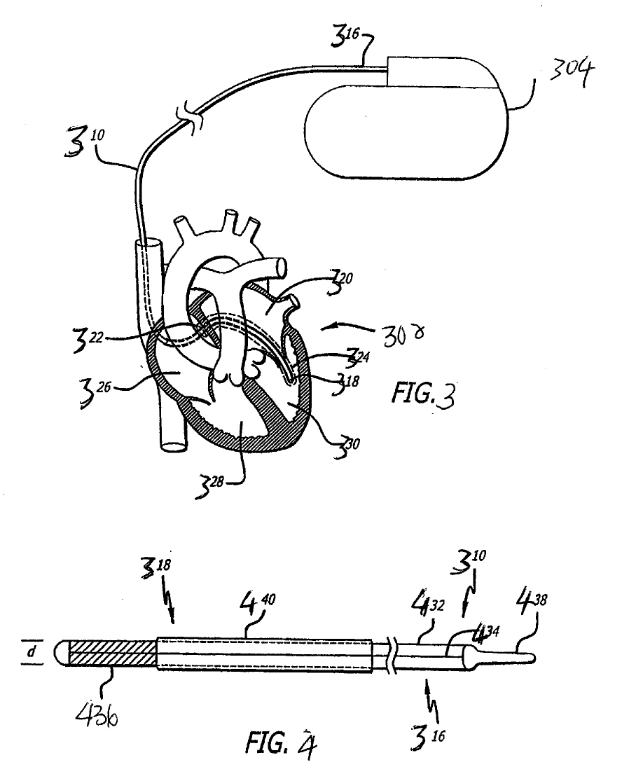

With reference to FIG. 3, in one embodiment, an implantable lead 310 provides for access to a chamber of a heart 302. Lead 310 is part of an implantable CRM device 304 and includes a proximal end 316, which is coupled to device 304, and a distal end portion 318, which is coupled on or about one or more portions of heart 302. In the illustrated embodiment, lead 310 is a coronary sinus lead. Device 304 may be implanted in response to a myocardial infarction and lead 310 may be positioned to provide access to a myocardial region in or near an infarct. In other embodiments, lead 310 is an epicardial lead providing access to an epicardial region of heart 302, or an endocardial lead providing access to an endocardial region of heart 302.

As illustrated in FIG. 3, distal end portion 318 of lead 310 is transvenously guided to a left ventricle 330, through a coronary sinus 322 and into a great cardiac vein 324. This positioning of lead 310 is useful for delivering pacing and/or defibrillation energy to the left side of heart 302 such as for treatment of cardiac disorders requiring therapy delivered to the left side of heart 302. Other possible positions of distal portion 318 of lead 310 include insertion in to a right atrium 326 and/or a right ventricle 328, or transeptal insertion into a left atrium 320 and/or left ventricle 330.

With reference to FIG. 4, in one embodiment, lead 310 includes a biocompatible flexible insulating elongate body 432 (e.g., including a polymer such as medical grade silicone rubber) for transluminal (i.e., transvenous or transarterial) insertion and access within a living organism. In one embodiment, the slender elongate body 432 is tubular and has a peripheral outer surface of diameter d that is small enough for transluminal insertion into coronary sinus 322 and/or great cardiac vein 324. An elongate electrical conductor 434 is carried within the insulating elongate body 432. Conductor 434 extends substantially along the entire length between distal end portion 318 and proximal end 316 of lead 310, and this length is long enough for lead 310 to couple device 304, which is implanted pectorally, abdominally, or elsewhere, to desired locations within heart 302 for sensing intrinsic electrical heart activity signals or providing pacing/defibrillation-type therapy.

The elongate body 432 forms an insulating sheath covering around conductor 434. Conductor 434 is coupled to a ring or ring-like electrode 436 at or near distal end portion 318 of the elongate body 42. The conductor 434 is coupled to a connector 438 at or near the proximal end 316 of elongate body 432. Device 304 includes a receptacle for receiving connector 438, thereby obtaining electrical continuity between electrode 436 and device 304.

Electrode 436, or at least a portion thereof, is not covered by the insulating sheath of elongate body 432. Electrode 436 provides an exposed electrically conductive surface around all, or at least part of, the circumference of lead 310. In one example, electrode 436 is a coiled wire electrode that is wound around the circumferential outer surface of lead 310. Lead 310 also includes other configurations, shapes, and structures of electrode 436.

As illustrated in FIG. 4, lead 310 includes a biocompatible coating 440 on at least one insulating portion of the peripheral surface of elongate body 436 at or near distal end portion 318. Coating 440 extends circumferentially completely (or at least partially) around the tubular outer peripheral surface of lead 310 and carries one or more inhibitory agents 212. In use, when lead 310 is inserted and implanted in the body, coating 40 dissolves and one or more inhibitory agents 212 are released. The time it takes for the coating 40 to fully dissolve and thus for one or more inhibitory agents 212 to be completely released may be controlled based on the selection of the coating material and the concentration of the one or more agents 212.

### Stents

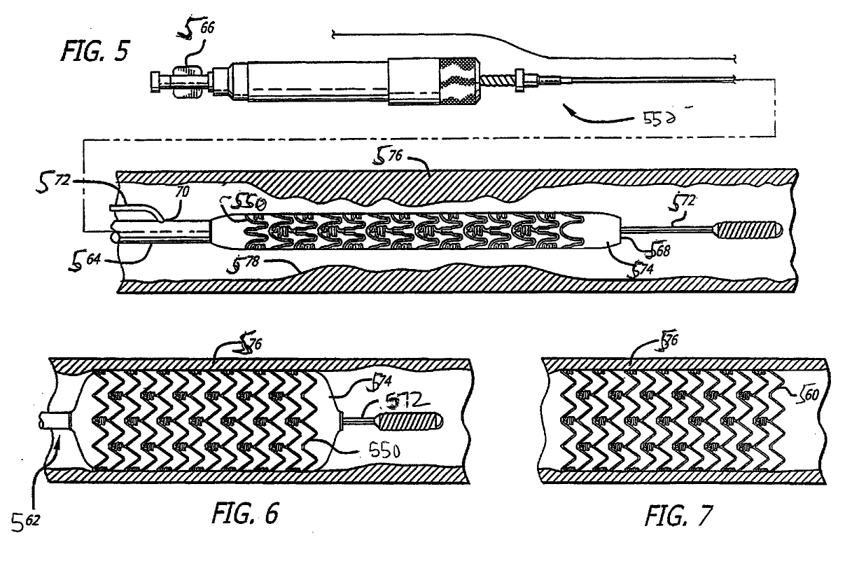

With reference to FIGS. 5, 6 and 7, in another embodiment, a stent 550 is positioned within the vascular system. As shown in FIG. 5, stent 550 is mounted on a catheter assembly 552 which is used to deliver stent 550 to implant it in a body lumen, such as a coronary artery, peripheral artery, or other vessel or lumen within the body. In one embodiment, stent 550 is formed of a metallic material. In another embodiment, stent 550 is formed of a polymer material. Catheter assembly 552 includes a catheter shaft 564 which has a proximal end 566 and a distal end 568. Catheter assembly 552 is configured to advance through the patient's vascular system by advancing over a guide wire 572.

Catheter assembly 552 as illustrated in FIG. 5 is of a rapid exchange type which includes an RX port 570 where guide wire 572 will exit the catheter. The distal end of guide wire 572 exits catheter distal end 568 so that the catheter advances along the guide wire on a section of the catheter between RX port 570 and catheter distal end 568. The guide wire lumen which receives guide wire 572 is sized for receiving various diameter guide wires to suit a particular application. Stent 550 is mounted on expandable member 574 and is crimped tightly thereon so that stent 550 and expandable member 574 present a low profile diameter for delivery through the arteries.

In FIG. 5, a partial cross-section of an artery 576 is shown with a small amount of plaque that has been previously treated by an angioplasty or other repair procedure. Stent 550 is used to repair a diseased or damaged arterial wall which may include plaque 578 as shown in FIG. 5, or a dissection, or a flap which are sometimes found in the coronary arteries, peripheral arteries and other vessels.

In an exemplary procedure to implant stent 550, guide wire 572 is advanced through the patient's vascular system by well known methods so that the distal end of the guide wire is advanced past the plaque or diseased area 578. Prior to implanting stent 550, the cardiologist may wish to perform an angioplasty procedure or other procedure, i.e., atherectomy, in order to open the vessel and remodel the diseased area. Thereafter, stent delivery catheter assembly 552 is advanced over guide wire 572 so that stent 550 is positioned in the target area. The expandable member or balloon 574 is inflated so that it expands radially outwardly and in turn expands stent 550 radially outwardly until stent 550 is apposed to the vessel wall. Expandable member 574 is then deflated and the catheter withdrawn from the patient's vascular system. Guide wire 572 is left in the lumen for post-dilatation procedures, if any, and subsequently is withdrawn from the patient's vascular system. As illustrated in FIG. 6, the balloon 574 is fully inflated with stent 550 expanded and pressed against the vessel wall, and in FIG. 7, stent 550 remains in the vessel after the balloon has been deflated and catheter assembly 552 (FIG. 6) and guide wire 572 have been withdrawn from the patient.

Stent 550 serves to hold open the artery after the catheter is withdrawn, as illustrated by FIG. 7. Due to the formation of stent 550 from an elongated tubular member, the undulating components of stent 550 are relatively flat in transverse cross-section. When stent 550 is expanded, it is pressed into the wall of the artery and accordingly does not interfere with the blood flow through the artery. Stent 550 is pressed into the wall of the artery and will eventually be covered with endothelial cell growth which further minimizes blood flow interference.

In one embodiment, the entire surface of stent 550 is coated to carry and deliver one or more inhibitory agents 212. In another embodiment, portions of the surfaces of the stent 550, e.g., the tissue contacting portions, are coated to carry the one or more inhibitory agents 212.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An implantable device for treating living tissue, comprising:
an intravascular device; and
a composition comprising one or more agents incorporated into at least a portion of the intravascular device, wherein the one or more agents comprise a vascular adhesion protein-1 (VAP-1) inhibitor.

2. The device of claim 1 wherein the intravascular device comprises a transvenous lead.

3. The device of claim 1 wherein the intravascular device comprises a stent.

4. The device of claim 1 wherein the one or more agents comprise a semicarbazide sensitive amine oxidase (SSAO) inhibitor.

5. The device of claim 1 wherein the one or more agent is a sustained release formulation.

6. The device of claim 1 wherein the agent comprises a hydrazine.

7. The device of claim 1 wherein the agent comprises an arylalkylamine.

8. The device of claim 1 wherein the agent comprises a propenylamine.

9. The device of claim 1 wherein the agent comprises a proparyl amine.

10. The device of claim 1 wherein the agent comprises an oxazolidinone.

11. The device of claim 1 wherein the agent comprises a haloalkylamine.

12. A method to inhibit or treat inflammation, restenosis, or oxidative stress in a mammal, comprising introducing to a mammal in need thereof an implantable device comprising a composition comprising an effective amount of one or more agents incorporated into at least a portion of the implantable device, wherein the one or more agents comprise a vascular adhesion protein-1 (VAP-1) inhibitor.

13. The method of claim 12 wherein the agent comprises a hydrazine.

14. The method of claim 12 wherein the agent comprises an arylalkylamine.

15. The method of claim 12 wherein the agent comprises a propenylamine.

16. The method of claim 12 wherein the agent comprises a proparylamine.

17. The method of claim 12 wherein the agent comprises an oxazolidinone.

18. The method of claim 12 wherein the agent comprises a haloalkylamine.

19. The method of claim 12 wherein the mammal is a human.

20. The method of claim 12 wherein the mammal has diabetes.

21. The method of claim 12 wherein the implantable device is a intraluminal device including at least a portion configured to be placed in a body lumen.

22. The method of claim 21 wherein the intraluminal device is a stent.

23. The method of claim 21 wherein the intraluminal device is an intravascular device.

24. The method of claim 12 wherein the composition comprises a sustained release formulation.

25. The method of claim 12 wherein the amount is effective to inhibit or treat inflammation.

26. The method of claim 12 wherein the amount is effective to inhibit or treat restenosis.

27. The method of claim 12 wherein the amount is effective to inhibit or treat oxidative stress.
